(19) United States
(12) Patent Application Publication
(10) Pub. No.: US 2004/0185459 A1
(43) Pub. Date: Sep. 23, 2004

(54) Hybridization probe

(76) Inventors: Masami Otsuka, Kumamoto (JP); Tetsuo Yamasaki, Kumamoto (JP); Shigemichi Gunji, Tokyo (JP); Fujio Yu, Kanagawa (JP); Katsuaki Kikuchi, Kanagawa (JP); Toshitaka Uragaki, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/475,316

(22) PCT Filed: Apr. 18, 2001
(86) PCT No.: PCT/JP01/03322

(51) Int. Cl. C12Q 1/68; C07H 21/04

(52) U.S. Cl. 435/6; 536/24.3

(57) ABSTRACT

A probe is utilized in the hybridization with a natural nucleic acid to form a double strand. The probe contains a cytosine derivative which specifically binds to guanine by forming two hydrogen bonds with the guanine nucleotide and/or a guanine derivative which specifically binds to cytosine by forming two hydrogen bonds with the cytosine nucleotide. This enables a large number of hybridization reactions to be carried out at once under uniform conditions.

### TECHNICAL FIELD

This invention relates to a probe which is adapted for use in hybridization of complementary single strand nucleic acids into a double strand nucleic acid.

### BACKGROUND ART

Hybridization is a reaction based on denaturing of double strand nucleic acid and association of complementary strands. Since hybridization takes place between complementary strands, hybridization is utilized in purification and analysis of the nucleic acids.

An analysis utilizing the hybridization reaction is basically carried out by preparing the analyte sample containing the target sequence, and hybridizing a labeled probe which is complementary to the target sequence with the target sequence to thereby screen the target sequence that became hybridized with the labeled probe. A wide variety of analyses utilizing the hybridization are conducted for various applications, and the analyses vary by the method used for preparing the analyte sample, the type of the probe (a cloned DNA or a synthetic nucleic acid) used, the method used for the labeling, the means used for the analysis, and the like.

For example, southern hybridization and northern hybridization are methods wherein DNA of the cloned gene is labeled for use as a probe, and DNA or mRNA of the gene from various tissues or cells are used for the analyte sample to confirm and/or quantitate the complementary or analogous genes.

PCR is also basically a combination of hybridization reaction with synthetic oligo DNA primers and DNA amplification reaction.

Another noteworthy analysis utilizing the hybridization is the analysis using a DNA chip (or DNA microarray) which is capable of analyzing a large number of genes of a particular group at once. DNA chip is a piece of flat surface substrate of one to several cm² on which a large number of DNA fragments have been aligned and immobilized at a high density. The DNA chip may be the one having oligo nucleic acids of substantially same length chemically synthesized in situ on the substrate, or the one having natural cDNAs immobilized thereon. In the analysis using a DNA microarray, the analysis is conducted by amplifying the expression gene of the cell of interest or the like by an appropriate means, labeling the amplified gene with a fluorescent substance or the like, and allowing the labeled gene to hybridize with the probes that had been immobilized on the flat substrate, and scanning the surface of the chip with a high speed laser scanner or the like to thereby quantitate the expression of the genes of several to several dozen thousand varieties at once, or conduct a relative comparison of the expression between the specimens. In the case of an oligo nucleic acid, detection of a mutation as well as sequencing of the particular gene can be accomplished by arranging a set of probes each having unique sequence (SBH: Sequencing by Hybridization. Drmanac, R. et al. Genomics 4: 114-128 (1989); Drmanac, R. et al. Science 260: 1649-1652 (1993)).

[Problem to be Solved]

In the analysis utilizing the hybridization, the extent of mismatch a probe may have in complementarity with target sequence so that the probe hybridizes with the target sequence differs depending on the conditions such as reaction temperature and salt concentration used in the hybridization. Therefore, the hybridization conditions are set depending on the intended stringency (degree of allowing for the mismatch). The hybridization conditions are usually selected by considering the melting temperature Tm between the probe and the target sequence. The Tm, however, is known to depend on the nucleotide composition, and contents of the guanine nucleotide (G) and the cytosine nucleotide (C) in the hybridized region.

However, when a large number of hybridization reactions should be conducted at once under the same conditions as in the case of analysis using a DNA chip, the only effective means had been exclusion of inadequate probes and use of probes having a uniform content of G and C. When the probes used are to have a uniform content of G and C, the region in the subject gene sequence that could be used for the probe inevitably became quite limited.

### DISCLOSURE OF THE INVENTION

There are two types of purine-pyrimidine base pair, namely, the guanine-cytosine base pair involving three hydrogen bonds and the adenine-thymine (uracil in the case of an RNA) base pair involving two hydrogen bonds. Present inventers considered it would be possible to unify values characteristic to hybridization such as the value of Tm (melting temperature) which differs depending on probe sequence, by using nucleic acid base derivatives capable of forming an equal number of hydrogen bonds without substantial influence on the binding specificity between the particular base pair in the formation of purine-pyrimidine base pairs. An object of the present invention is to provide a method which has enabled to carry out a large number of hybridization reactions at once under uniform conditions without paying attention to the difference in the nucleotide sequence of the probes.

Hybridization reactions can be carried out at once under uniform conditions when a guanine derivative capable of specifically binding to cytosine by forming two hydrogen bonds with the cytosine nucleotide, and a cytosine derivatives capable of specifically binding to guanine by forming two hydrogen bonds with the guanine nucleotide are synthesized, and the probes containing such derivatives are used for the hybridization reactions.

### BEST MODE FOR CARRYING OUT THE INVENTION

Next, the present invention is described in detail.

The prove of the present invention is a probe used in the hybridization reaction with a natural nucleic acid, and contains a cytosine derivative which specifically binds to guanine by forming two hydrogen bonds with the guanine nucleotide and a guanine derivative which specifically binds to cytosine by forming two hydrogen bonds with the cytosine nucleotide. The prove of the present invention is also characterized in that, in the hybridization, substantially all base pairs hybridize by forming two hydrogen bonds to the extent such that the value of Tm can be regarded identical in terms of the conditions of hybridization. The phrase “substantially all” means that at least 80%, preferably at least 95%, and more preferably all of the C and G have been replaced with the guanine derivative and the cytosine derivative as described above.

The cytosine derivative used in the present invention is a compound represented by any one of the following formulae: (I) to (V), each having a structure wherein at least one of the three moieties in the cytosine capable of forming hydrogen bond with guanine (that is, amino group at position 4, nitrogen at position 3, and ketone at position 2) has been modified to prevent the formation of the hydrogen bond. Exemplary such compounds include the compounds of formulae (I) and (II) wherein formation of the hydrogen bond at the amino group at position 4 has been avoided, the compound of formula (III) wherein formation of the hydrogen bond at the nitrogen at position 3 has been avoided, and the compound of formulae (IV) and (V) wherein formation of the hydrogen bond at the ketone at position 2 has been avoided.

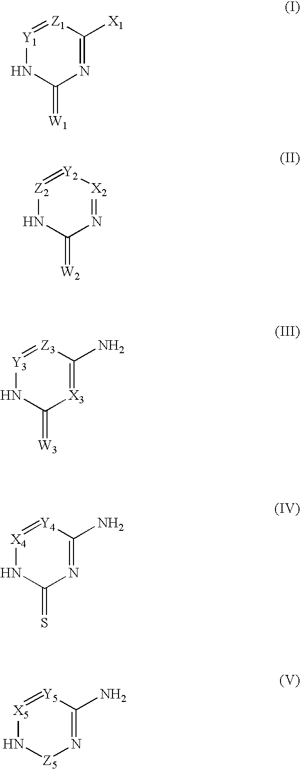

In the formulae, X₁ represents NR₂, NHAc, R, OR, OAc, SR, SAc, COR, COOR, CN, F, Cl, Br, or I; W₁, W₂, and W₃ represent O or NH; X₃ represents CH or CR; Z₅ represents CH₂ or CHR; Y₁, Z₁, X₂, Y₂, Z₂, Y₃, Z₃, X₄, Y₄, X₅, and Y₅ represent CH, CR, or N; with the proviso that R represents a substituent which does not inhibit the two hydrogen bonds formed between the cytosine derivative and the guanine.

The guanine derivative used in the present invention is a compound represented by any one of the following formulae: (VI) to (X), each having a structure wherein at least one of the three moieties in the guanine capable of forming hydrogen bond with cytosine (that is, amino group at position 2, nitrogen at position 1, and ketone at position 6) is prevented from forming the hydrogen bond. Exemplary such compounds include the compounds of formulae (VI) and (VII) wherein formation of the hydrogen bond at the amino group at position 2 has been avoided, the compound of formula (VIII) wherein formation of the hydrogen bond with the nitrogen at position 1 has been avoided, and the compound of formulae (IX) and (X) wherein formation of the hydrogen bond at the ketone at position 6 has been avoided.

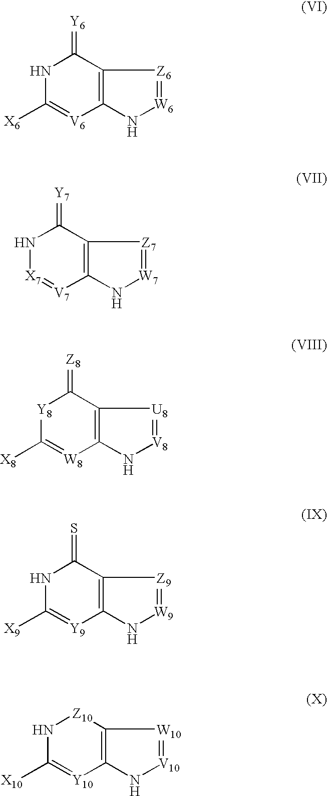

In the formulae, X₆ and X₈ represent NR₂, NHAc, R, OR, OAc, SR, SAc, COR, COOR, CN, F, Cl, Br, or I; Y₆, Y₇, and Z₈ represent O or NH; Y₈ and Z₁₀ represent CH₂, CHR, O, or S; X₉ and X₁₀ represent NH₂ or OH; and V₆, W₆, Z₆, V₇, W₇, X₇, Z₇, U₈, V₈, W₈, Wg, Y₉, Z₉, V₁₀, W₁₀, and Y₁₀ represent CH, CR, or N with the proviso that R represents a substituent which does not inhibit the two hydrogen bonds between the cytosine and the guanine derivative.

The cytosine derivative and guanine derivative as described above can be synthesized by the method commonly used in the art.

The backbone of the probe of the present invention is not particularly limited as long as it is capable of undergoing hybridization. Exemplary backbones include a DNA, an RNA, a peptide nucleic acid (a nucleic acid wherein the sugar-phosphate chain has been replaced with a charge-neutral peptide chain; J.Am.Chem.Soc. 114, 1985 (1992)), and a nucleic acid analog called LNA (a nucleic acid analog wherein methylene group has been introduced between the oxygen at position 2 and the carbon at position 4 of the furanose ring constituting the nucleic acid nucleoside; Bioconjug. Chem. 1 (2) 228-38 (2000)). The probe may be produced by using an automated nucleic acid synthesizer or an automated peptide synthesizer by a method commonly used in the art.

In the present invention, the term “probe set” designates a set or a group of probes. The probe set may be prepared in accordance with the intended purpose of the assay, and exemplary such probe sets include a probe set for detecting cancer related genes, a probe set for detecting diabetes related genes, a probe set for detecting genes of microorganism, yeast, vegetable, and other biological species.

In the present invention, the probe set may be immobilized on an appropriate carrier such as a resin, a glass bead, or a gel so that each probe is identifiable, or arranged in an array on a substrate to constitute a DNA chip.

In the present invention, occurrence of the hybridization reaction can be confirmed by any method commonly used in the art. For example, in the case of the hybridization of the probe with a complementary DNA, occurrence of the hybridization may be confirmed by measuring UV absorption while altering the temperature. In such a case, melting temperature (Tm) may also be determined from the inflection point of the UV absorption curve. In the case of the probes incorporated in a DNA chip, the hybridization may be confirmed by preparing mRNA from the sample of a particular organism, preparing cDNA from the mRNA using a reverse transcriptase, labeling the cDNA with fluorescence to produce a fluorescence-labeled specimen (hereinafter referred to as the labeled specimen), incubating the labeled specimen in SSC buffer at 50 to 60° C. for 10 to 20 hours on the DNA chip, washing the DNA chip, and detecting the hybridized spot by using a scanner for microarray or the like.

The probe of the present invention can be used for a gene expression analysis or detection by a DNA chip which is capable of treating a large number of samples at once, and also, for an SNP analysis whose future importance has been pointed out, and for a gene sequence analysis by hybridization (SBH).

### EXAMPLES

Next, the present invention is further described in detail by referring to Examples which by no means limit the scope of the invention.

### Example 1

### Synthesis of Nucleic Acid-Type Probe

(1) Synthesis of Cytosine Derivative (deoxyribose-6-aza-3-deazacytosine phosphoroamidite)

Anhydrous hydrazine is reacted with mucochloric acid to synthesize dichloropyridazinone. Chloro group at the position 4 is aminated with ammonia to synthesize compound 1.

To the suspension of compound 1 (2.2 g) in methanol (90 ml)-dimethylformamide (90 ml) is added sodium hydroxide (0.604 g) and 10% palladium carbon (0.9 g), and the mixture is stirred at normal pressure for 7 days while purging with hydrogen gas. Palladium carbon is filtered off, and the reaction solvent is distilled off under reduced pressure. The resulting residue is recrystallized in purified water to obtain compound 2 (0.835 g).

The suspension of compound 2 (1.0 g) in anhydrous pyridine (50 ml) is cooled in an ice bath, and benzoyl chloride (2.09 ml) is added dropwise while purging the system with argon gas. The mixture is stirred at room temperature for one day while purging with argon gas, and the reaction medium is distilled off under reduced pressure. Purified water (10 ml) is added to the resulting residue, and 4M hydrochloric acid is added to adjust the pH to 1. Precipitated crystals are collected by filtration, and washed with purified water. After drying under reduced pressure, anhydrous ethanol (10 ml) is added to the crystals. After boiling for about 10 minutes and cooling to 10° C., the crystals are collected by filtration and washed with ether to obtain compound 3 (6-aza-deazacytosine, 1.399 g).

To the suspension of compound 3 (1.00 g) and potassium carbonate (0.70 g) in dimethylformamide (12.9 ml) is added 1-chlorodeoxyribose protected by p-toluoyl (2.0 g) which had been separately synthesized by an ordinary process, and the mixture is stirred at room temperature for 2 days while purging with argon gas. The insoluble content is removed by filtration, and the reaction solvent is distilled off from the filtrate under reduced pressure. Purified water (5 ml) is added to the residue, and 4M hydrochloric acid (0.175 ml) is added in an ice bath. After stirring for 15 minutes, crystals are collected by filtration, washed with purified water, and compound 4 (2.8 g) was obtained as a mixture of α and β forms. Compound 4 was then subjected to column chromatography by using a column having 150 g of wakogel C-200 filled therein, and adding methylene chloride-ethyl acetate mixed solvent to the column for fractionation. The fractions containing the β form are collected and concentrated to obtain compound 5 (1.2 g).

Compound 5 (1.0 g) is mixed with a suspension of potassium carbonate (0.6 g) in dimethylformamide (11 ml), and the mixture is stirred at 30° C. for 1 hour. The insoluble content is removed by filtration, and the filtrate is concentrated under reduced pressure. Purified water (5 ml) is added to the residue, and 4M hydrochloric acid (0.1 ml) is added in an ice bath. After stirring for 15 minutes, the precipitated crystals are collected by filtration and recrystallized from methanol to obtain compound 6 (0.4 g) Compound 6 (0.3 g) is azeotropically dehydrated with anhydrous pyridine under reduced pressure, and dissolved in anhydrous pyridine (1 ml). 4,4′-dimethoxytrityl bromide (0.6 g) is added to the solution, and the mixture is stirred at 60° C.s. Heating is stopped after 2 hours, and after returning of the mixture to room temperature, water (2 ml) is added to the mixture, and extraction with dichloromethane (2 ml) is conducted twice followed by drying and concentration. The residue is azeotroped twice with toluene to completely remove pyridine. Toluene is added to wash the precipitated crystals, and the crystals are dried under reduced pressure to produce compound 7 (0.5 g) which is protected by trityl.

Compound 7 (0.4 g) is azeotropically dehydrated twice with anhydrous pyridine and twice with dry toluene under reduced pressure, and dissolved in dichloromethane (5 ml). To the solution is added diisopropylethylamine (1.6 ml), and the solution is cooled to 0° C. To the solution is added chloro-2-cyanoethoxydiisopropylaminophosphine (0.3 g), and the mixture is stirred at room temperature. After 1 hour, dichloromethane (5 ml) is added, and washed twice with 5% aqueous solution of sodium bicarbonate (3 ml), dried over anhydrous sodium sulfate, and the residue after the concentration is purified by silica gel chromatography to obtain deoxyribose-6-aza-3-deazacytosine phosphoroamidite (0.5 g) (compound 8).

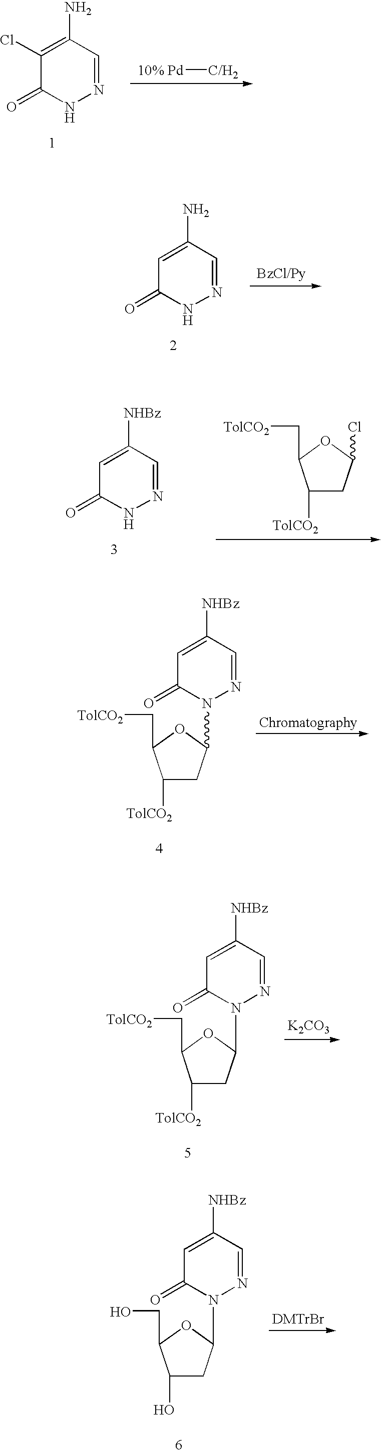

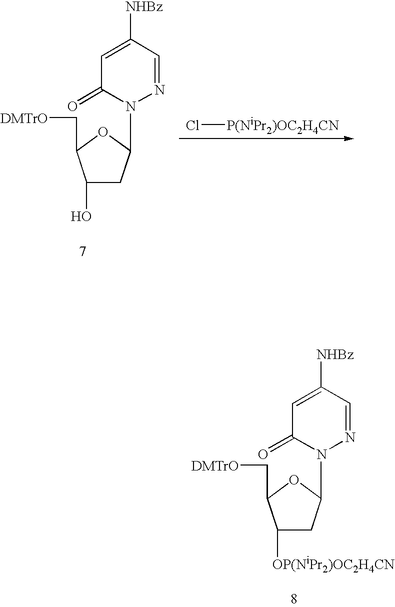

Bz: benzoyl group; Tol: tolyl group; DMTr: dimethoxytrityl group

(2) Synthesis of Guanine Derivative (deoxyribose oxanine phosphoroamidite)

Oxanocine having deoxyribose in the sugar moiety is synthesized by the method described in the document (Tetrahedron Letters 24, 931 (1983)).

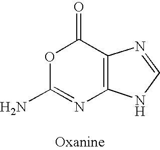

This deoxyribose oxanine (0.5 g) (compound 9) is azeotropically dehydrated with anhydrous pyridine under reduced pressure, and dissolved in anhydrous pyridine (5 ml). To this solution is added 4,4′-dimethoxytrityl bromide (0.6 g), and the mixture is stirred at 60° C. After 2 hours, heating is stopped, and after returning of the mixture to room temperature, water is added to the mixture, and extraction with dichloromethane (5 ml) is conducted twice followed by drying and concentration. The residue is azeotroped twice with toluene to completely remove pyridine. Toluene is added to wash the precipitated crystals, and the crystals are dried under reduced pressure to produce compound 10 (0.8 g) which is protected by trityl.

The compound protected with trityl (0.7 g) is azeotropically dehydrated twice with anhydrous pyridine and twice with dry toluene under reduced pressure, and dissolved in dichloromethane (5 ml). To this solution is added diisopropylethylamine (2.0 ml), and the mixture is cooled to 0° C. To the solution is added chloro-2-cyanoethoxydiisopropylaminophosphine (1.0 g), and the mixture is stirred at room temperature. After 1 hour, dichloromethane (5 ml) is added, and washed twice with 5% aqueous solution of sodium bicarbonate (3 ml), dried over anhydrous sodium sulfate, and the residue after the concentration is purified by silica gel chromatography to obtain deoxyribose oxanine phoshoramidite (0.9 g) (compound 11).

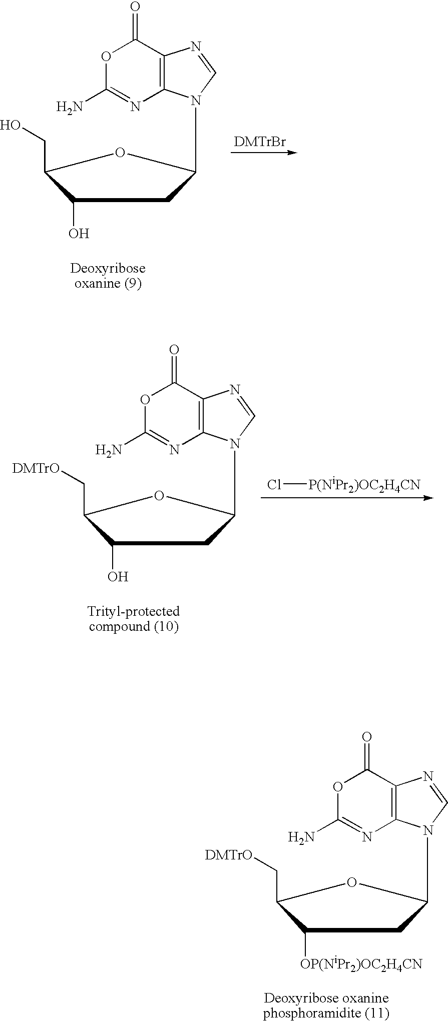

(3) Preparation of Probe

The thus synthesized cytosine derivative phoshoroamidite (deoxyribose-6-aza-3-deazacytosine phorphoroamidite) (compound 8) and guanine derivative phoshoroamidite (deoxyribose oxanine phoshoroamidite) (compound 11) are used to synthesize an oligonucleotide. The synthesis of the oligonucleotide is carried out by using an automated DNA/RNA synthesizer (model 394) manufactured by PE Biosystems Inc.

### Example 2

### Synthesis of Peptide Nucleic Acid

(1) Synthesis of Cytosine Derivative (Compound 15)

To the suspension of compound 1 (2.2 g) in methanol (90 ml)-dimethylformamide (90 ml) is added sodium hydroxide (0.604 g) and 10% palladium carbon (0.9 g), and the mixture is stirred at normal pressure for 7 days while purging with hydrogen gas. Palladium carbon is removed by filtration, and the reaction medium is distilled off under reduced pressure. The resulting residue is recrystallized from purified water to obtain compound 2 (0.835 g).

The suspension of compound 2 (1.0 g) in anhydrous pyridine (50 ml) is cooled in an ice bath, and benzoyl chloride (2.09 ml) is added dropwise while purging with argon gas. The mixture is stirred at room temperature for one day while purging with argon gas, and the reaction solvent is distilled off under reduced pressure. Purified water (10 ml) is added to the resulting residue, and 4M hydrochloric acid is added to adjust the pH to 1. The precipitated crystals are collected by filtration, and washed with purified water. After drying under reduced pressure, anhydrous ethanol (10 ml) is added to the crystals, and the crystals are boiled for 10 minutes. After cooling to 10° C., the crystals are collected by filtration and washed with ether to obtain compound 3 (1.399 g).

To the suspension of compound 3 (1.00 g) and potassium carbonate (0.70 g) in dimethylformamide (12.9 ml) is added methyl bromoacetate (0.48 ml), and the mixture is stirred at room temperature for 2 days while purging with argon gas. The insoluble content is removed by filtration, and the reaction solvent in the filtrate is distilled off under reduced pressure. To the resulting residue is added purified water (4.5 ml), and then 4M hydrochloric acid (0.175 ml) is added in a ice bath, and mixture is stirred for 15 minutes. The crystals are collected by filtration, and washed with purified water to obtain methyl ester (compound 12). To this compound 12 are added purified water (6.75 ml) and 2M sodium hydroxide (3.38 ml), and the mixture is stirred for 30 minutes. The reaction solution is cooled to 0° C., and the insoluble content is removed by filtration. 4M hydrochloric acid (1.97 ml) is added to the filtrate and the precipitated crystals are collected by filtration. The collected crystals are recrystallized from methanol to obtain compound 13 (0.779 g). To the solution of compound 13 (0.601 g), ter-Butyl N-[2-(N-9-fluorenylmethoxycarbonyl)aminoethyl]glycinate (1.047 g), HOBt (0.337 g), and DIEA (0.766 ml) in DMF (20 ml) is added TBTU (0.706 g), and the mixture is stirred at room temperature for 12 hours. After distilling off the reaction solvent under reduced pressure, dichloromethane (150 ml) is added to the residue, and washed with purified water (100 ml×3), 4% aqueous solution of sodium hydrogencarbonate (100 ml×3), 4% aqueous solution of potassium hydrogensulfate (100 ml×3), and purified water (100 ml×3) in this order. The dichloromethane layer is dried over magnesium sulfate, and the solvent is distilled off under reduced pressure. The resulting crystals are recrystallized from ethyl acetate-n-hexane mixed solvent to obtain compound 14 (1.31 g).

Compound 14 (0.30 g) is added to dichloromethane (3 ml) −TFA (4 ml) mixed solvent, and the mixture is stirred at 0° C. for 30 minutes, and at room temperature for another 3 hours. The reaction solvent is distilled off under reduced pressure, and dry ether (5 ml) is added. The precipitated crystals are recrystallized from ethyl acetate-n-hexane mixed solvent to obtain compound 15 (0.256 g).

DIEA: diisopropylethylamine

TFA: trifluoroacetic acid

HOBt: 1-hydroxy-1H-benzotriazol

TBTU:

2-(1H-benzotriazol-1-yl)-1,1,3,3-tetramethyluronium tetrafluoroborate

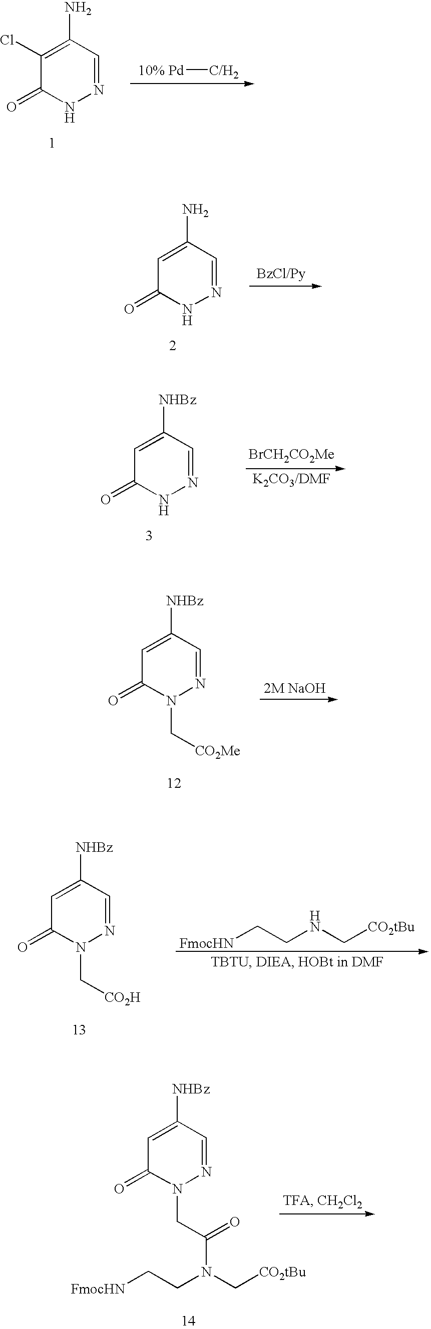

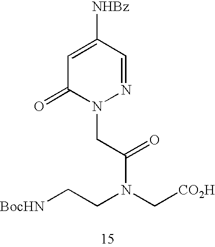

(2) Synthesis of Guanine Derivative (Compound 21)

To the suspension of 5-amino-3H-imidazo[4,5-d][1,3]oxazin-7-one 16 and potassium carbonate in dimethylformamide is added methyl bromoacetate, and the mixture is stirred at room temperature for 24 hours while purging with argon gas. The insoluble content is removed by filtration, and the reaction solvent in the filtrate is distilled off under reduced pressure. Purified water is added to the resulting residue, and the precipitated crystals are collected by filtration. The crystals are recrystallized from dimethylformamide-ethanol mixed solvent to obtain methyl ester (compound 17).

The suspension of compound 17 in anhydrous pyridine is cooled in a water bath, and benzoyl chloride is added dropwise while purging with argon gas. The mixture is stirred at room temperature for one day while purging with argon gas, and the reaction solvent is distilled off under reduced pressure. Purified water is added to the residue, and 1M aqueous solution of hydrochloric acid is added in an ice bath to adjust the pH to 1. The precipitated crystals are collected by filtration, and recrystallized from dimethylformamide-ethanol mixed solvent to obtain compound 18.

Compound 18 is added to 2M aqueous solution of sodium hydroxide, and the mixture is stirred at 30 minutes. The reaction solution is cooled to 0° C., and the insoluble content is removed by filtration. To the filtrate is added 4M aqueous solution of hydrochloric acid in an ice bath to adjust the pH to 3, and the precipitated crystals are collected by filtration. The precipitated crystals are recrystallized from dimethylformamide-ethanol mixed solvent to obtain compound 19. To the solution of compound 19, ter-Butyl N-[2-(N-9-fluorenylmethoxycarbonyl)aminoethyl]glycinate, HOBt, and DIEA in DMF is added TBTU, and the mixture is stirred at room temperature for 12 hours. After distilling off the reaction solvent under reduced pressure, dichloromethane (200 ml) is added to the residue, and washed with purified water (100 ml×3), 4% aqueous solution of sodium hydrogencarbonate (100 ml×3), 4% aqueous solution of potassium hydrogensulfate (100 ml×3), and purified water (100 ml×3) in this order. After drying the dichloromethane layer over magnesium sulfate and distilling off under reduced pressure, the resulting crystals are recrystallized from ethanol-n-hexane mixed solvent to obtain compound 20. Compound 20 is added to the dichloromethane-TFA mixed solvent, and the mixture is stirred at 0° C. for 30 minutes, and at room temperature for another 3 hours. After distilling of the reaction solvent under reduced pressure, dry ether (5 ml) is added and the precipitated crystals are recrystallized from ethanol-n-hexane mixed solvent to obtain compound 21.

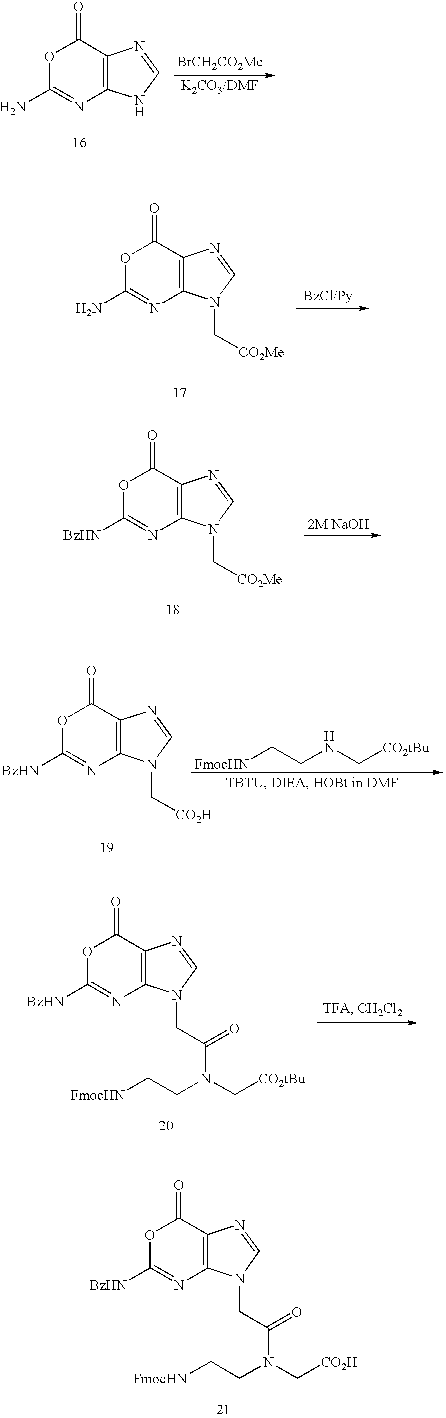

(3) Preparation of Probe

Compound 15 and compound 12 are used to synthesize an oligopeptide nucleic acid. The synthesis of the oligopeptide nucleic acid is carried out by using manual personal organic synthesizer CCS-600V manufactured by Tokyo Rikakikai K.K.

### Example 3

### Hybridization

[Nucleic Acid-Type Probe: Cytosine Derivative and Guanine Derivative]

Two oligo DNAs (A′ and B′) corresponding to two types of oligo DNAs (oligo DNA A and oligo DNA B) are prepared by using 6-aza-3-deazacytosine instead of cytosine and oxanine instead of guanine in the oligo DNA A and oligo DNA B.

Oligo DNA E wherein the contents of G and C are lower than those of oligo DNAs A and B is prepared. Oligo DNA E′ wherein cytosine had been replaced with 6-aza-3-deazacytosine and guanine had been replaced with oxanine in oligo DNA E is also produced.

Oligo DNA F which is complementary to oligo DNA E is also produced.

Oligo DNA A: atgccacgctatccgatgcc

Oligo DNA A′: ateddadedtatddeatedd

Oligo DNA B: atgcgacggtatcggatgcg

Oligo DNA B′: atedeadeetatdeeatede

Oligo DNA C: ggcatcggatagcgtggcat

Oligo DNA D: cgcatccgataccgtcgcat

Oligo DNA E: atgacactgtatccaatgac

Oligo DNA E′: ateadadtetatddaatead

Oligo DNA F: gtcattggatacagtgtcat

(a, t, c, and g represent adenine, thymine, cytosine, and guanine, respectively. d represents 6-aza-3-deazacytosine, and e represents oxanine.)

When the melting temperature of the double stranded DNA of oligo DNA A′ (the DNA replaced with 6-aza-3-deazacytosine and oxanine)/oligo DNA C is examined by measuring UV absorption, it is lower than the melting temperature of oligo DNA A/oligo DNA C. The melting temperature of the double stranded DNA of oligo DNA B′/oligo DNA D measured is also lower than the melting temperature of oligo DNA B/oligo DNA D. The melting temperature of the double stranded DNA of oligo DNA A′/oligo DNA C is measured to be equivalent to the melting temperature of the double stranded DNA of oligo DNA B′/oligo DNA D.

The melting temperature of the double stranded DNA of oligo DNA E′ (the DNA replaced with 6-aza-3-deazacytosine and oxanines)/oligo DNA F is measured to be lower than the melting temperature of double stranded DNA of oligo DNA E/oligo DNA F. The melting temperature of the double stranded DNA of oligo DNA A′/oligo DNA C is measured to be equivalent to the melting temperature of the double stranded DNA of oligo DNA B′/oligo DNA D despite the considerable difference in the contents of G and C.

[Peptide Nucleic Acid: Cytosine Derivative and Guanine Derivative]

Two oligopeptide nucleic acids (A′ and B′) corresponding to two types of oligopeptide nucleic acids (oligopeptide nucleic acid A and oligopeptide nucleic acid B) are prepared by using 6-aza-3-deazacytosine instead of cytosine and oxanine instead of guanine in the oligopeptide nucleic acid A and oligopeptide nucleic acid B.

Oligopeptide nucleic acid E wherein the contents of G and C are lower than those of oligopeptide nucleic acids A and B is prepared. Oligopeptide nucleic acid E′ wherein cytosine had been replaced with 6-aza-3-deazacytosine and guanine had been replaced with oxanine in oligopeptide nucleic acid E is also produced.

Oligopeptide nucleic acid F which is complementary to oligopeptide nucleic acid E is also produced.

Oligopeptide nucleic acid A: atgccacgctatccgatgcc

Oligopeptide nucleic acid A′: ateddadedtatddeatedd

Oligopeptide nucleic acid B: atgcgacggtatcggatgcg

Oligopeptide nucleic acid B′: atedeadeetatdeeatede

Oligo DNA C: ggcatcggatagcgtggcat

Oligo DNA D: cgcatccgataccgtcgcat

Oligopeptide nucleic acid E: atgacactgtatccaatgac

oligopeptide nucleic acid E′: ateadadtetatddaatead

Oligo DNA F: gtcattggatacagtgtcat

(a, t, c, and g represent adenine, thymine, cytosine, and guanine, respectively. d represents 6-aza-3-deazacytosine, and e represents oxanine.)

When the melting temperature of the double strand of oligopeptide nucleic acid A′ (the peptide nucleic acid replaced with 6-aza-3-deazacytosine and oxanine)/oligo DNA C is examined by measuring UV absorption, it is lower than the melting temperature of oliopeptide nucleic acid A/oligo DNA C. The melting temperature of the double strand of oligopeptide nucleic acid B/oligo DNA D measured is also lower than the melting temperature of oligopeptide nucleic acid B′/oligo DNA D. The melting temperature of the double strand of oligopeptide nucleic acid A′/oligo DNA C is measured to be equivalent to the melting temperature of the double strand of oligopeptide nucleic acid B′/oligo DNA D.

The melting temperature of the double strand of oligopeptide nucleic acid E′ (the peptide nucleic acid replaced with 6-aza-3-deazacytosine and oxanines)/oligo DNA F is measured to be lower than the melting temperature of double strand of oligopeptide nucleic acid E/oligo DNA F. The melting temperature of the double strand of oligopeptide nucleic acid A′/oligo DNA C is measured to be equivalent to the melting temperature of the double strand of oligopeptide nucleic acid B′/oligo DNA D despite the considerable difference in the contents of G and C.

1. A probe containing a cytosine derivative capable of specifically binding to guanine nucleotide by forming two hydrogen bonds and a guanine derivative capable of specifically binding to cytosine nucleotide by forming two hydrogen bonds, wherein substantially all nucleotides in the probe are capable of forming two hydrogen bonds in the hybridization with a natural nucleic acid.

2. A probe according claim 1 wherein
the cytosine derivative is a compound represented by any one of the following formulae: (I) to (V): 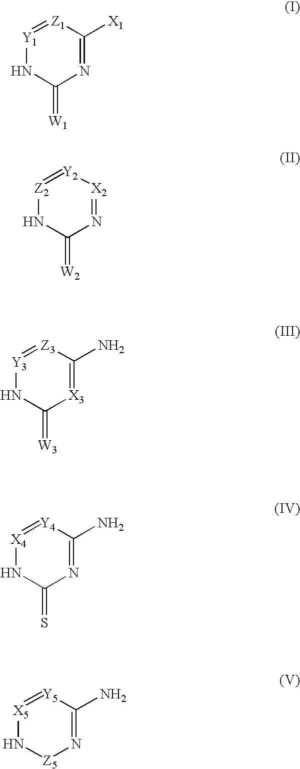
wherein X₁ represents NR₂, NHAc, R, OR, OAc, SR, SAc, COR, COOR, CN, F, Cl, Br, or I; W₁, W₂, and W₃ represent O or NH;
X₃ represents CH or CR; Z₅ represents CH₂ or CHR; Y₁, Z₁, X₂, Y₂, Z₂, Y₃, Z₃, X₄, Y₄, X₅, and Y₅ represent CH, CR, or N; with the proviso that R represents a substituent which does not inhibit the two hydrogen bonds formed between the cytosine derivative and guanine; and
the guanine derivative is a compound represented by any one of the following formulae: (VI) to (X): 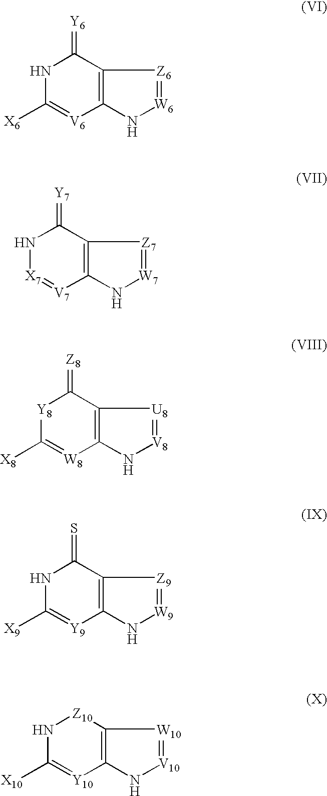
wherein X₆ and X₈ represent NR₂, NHAc, R, OR, OAc, SR, SAc, COR, COOR, CN, F, Cl, Br, or I; Y₆, Y₇, and Z₈ represent O or NH; Y₈ and Z₁₀ represent CH₂, CHR, O, or S; X₉ and X₁₀ represent NH₂ or OH; and V₆, W₆, Z₆, V₇, W₇, X₇, Z₇, U₈, V₈, W₈, W₉, Y₉, Z₉, V₁₀, W₁₀, and Y₁₀ respectively represent CH, CR, or N with the proviso that R represents a substituent which does not inhibit the two hydrogen bonds formed between cytosine and the guanine derivative.

3. A probe according to claim 1 or 2 wherein backbone of the probe is a deoxyribose phosphate chain.

4. A probe according to claim 1 or 2 wherein backbone of the probe is a peptide chain.

5. A probe set wherein all of the probes are selected from the probes of claims 1 to 4.

6. A carrier having probes immobilized thereon comprising the probe set of claim 5.

7. A DNA chip comprising the probe set of claim 5.

8. A method of hybridization using the probe set of claim 5, probe-immobilized carrier of claim 6 or the DNA chip of claim 7.

9. A method of SNP analysis using the hybridization method of claim 8.

10. A method for determining a DNA sequence using the hybridization method of claim 8.


SEQUENCE LISTING (9 sequences)

SEQ ID NO: 1 - DNA, 20 bp, Artificial Sequence
  synthetic oligonucleotide
atgccacgctatccgatgcc

SEQ ID NO: 2 - DNA, 20 bp, Artificial Sequence
  synthetic oligonucleotide
atnnnannntatnnnatnnn

SEQ ID NO: 3 - DNA, 20 bp, Artificial Sequence
  synthetic oligonucleotide
atgcgacggtatcggatgcg

SEQ ID NO: 4 - DNA, 20 bp, Artificial Sequence
  synthetic oligonucleotide
atnnnannntatnnnatnnn

SEQ ID NO: 5 - DNA, 20 bp, Artificial Sequence
  synthetic oligonucleotide
ggcatcggatagcgtggcat

SEQ ID NO: 6 - DNA, 20 bp, Artificial Sequence
  synthetic oligonucleotide
cgcatccgataccgtcgcat

SEQ ID NO: 7 - DNA, 20 bp, Artificial Sequence
  synthetic oligonucleotide
atgacactgtatccaatgac

SEQ ID NO: 8 - DNA, 20 bp, Artificial Sequence
  synthetic oligonucleotide
atnanantntatnnaatnan

SEQ ID NO: 9 - DNA, 20 bp, Artificial Sequence
  synthetic oligonucleotide
gtcattggatacagtgtcat